(19) United States
(12) Patent Application Publication
(10) Pub. No.: US 2013/0160788 A1
(43) Pub. Date: Jun. 27, 2013

(54) MULTI-PHASE LIQUID ANTIPERSPIRANT COMPOSITIONS AND ANTIPERSPIRANT PRODUCTS INCLUDING THE SAME

(71) Applicants: Debra K. Butterworth, Scottsdale, AZ (US); Travis T. Yarlagadda, Phoenix, AZ (US); Haiyan Ge, Scottsdale, AZ (US)

(72) Inventors: Debra K. Butterworth, Scottsdale, AZ (US); Travis T. Yarlagadda, Phoenix, AZ (US); Haiyan Ge, Scottsdale, AZ (US)

(73) Assignee: The Dial Corporation, Scottsdale, AZ (US)

(21) Appl. No.: 13/334,424

(22) Filed: Dec. 22, 2011

(51) Int. Cl. A45D 34/00; A61K 8/03

(52) U.S. Cl. 132/286; 424/65; 424/401; 206/581

(57) ABSTRACT

Multi-phase liquid antiperspirant compositions and antiperspirant products including multi-phase liquid antiperspirant compositions are provided. In one embodiment, a multi-phase liquid antiperspirant composition includes a first liquid phase, a second liquid phase, and a surfactant component. The first liquid phase includes an antiperspirant active compound and water. The second liquid phase includes a cyclomethicone component. The surfactant component is present in the multi-phase liquid antiperspirant composition in an amount sufficient to form an unstable emulsion of the first liquid phase and the second liquid phase upon mixing of the multi-phase liquid antiperspirant composition. The first liquid phase and the second liquid phase separate into visibly distinct layers within the multi-phase liquid antiperspirant composition after a settling time period of less than or equal to 48 hours. In another embodiment, an antiperspirant product includes a container and the multi-phase liquid antiperspirant composition disposed in the container.

### TECHNICAL FIELD

The present invention generally relates to multi-phase antiperspirant compositions and antiperspirant products including the multi-phase antiperspirant compositions. More particularly, the invention relates to multi-phase liquid antiperspirant compositions and antiperspirant products including the multi-phase liquid antiperspirant compositions.

### BACKGROUND

Antiperspirants and deodorants are popular personal care products used to prevent or eliminate perspiration and body odor caused by perspiration. Antiperspirants typically prevent the secretion of perspiration by blocking or plugging perspiration-secreting glands, such as those located at the underarms. Deodorants counteract or mask the unwanted odors caused by bacterial flora in secreted perspiration.

Antiperspirant compositions in stick form are desired by a large majority of the population because of the presence of antiperspirant active compounds that block or prevent the secretion of perspiration and the accompanying odors thereof and because of their ease of application. The antiperspirant compositions are applied to the skin by swiping or rubbing the stick across the skin, typically of the underarm. However, antiperspirant users often are disappointed in the chalky, brittle, and/or crumbly application of the stick across the skin.

Deodorant compositions in the form of water-based solutions have found wide commercial acceptance due to the ease of spray or roll-on application of the water-based solutions. Water-based solutions generally comprise a carrier of water and a co-solvent such as monohydric alcohol (for purposes of enhancing drying). While application of the deodorant compositions in the form of water-based solutions may be desired by certain consumers as an alternative to swipe application of antiperspirant compositions in stick form, the deodorant compositions do not prevent or eliminate the secretion of perspiration as do antiperspirants because they do not contain antiperspirant active compounds.

While a commercial desire may exist to provide antiperspirant compositions that are capable of spray application, there are challenges to distinguishing antiperspirant products that contain such antiperspirant compositions from the plethora of commercially available deodorant products. In particular, antiperspirant products and deodorant products are generally displayed to consumers in a common area in retail outlets, and it is challenging to impress upon consumers the value added by the antiperspirant products as compared to the deodorant products under such circumstances. Further, based upon the plethora of commercially available deodorant products and antiperspirant products that are on the market, antiperspirant products containing antiperspirant compositions that are visibly distinct from other commercially available deodorant and antiperspirant compositions may possess a competitive advantage. Further still, the marketplace has shown an increasing acceptance of and, in fact, has shown a desire for products that appear more clinical in nature.

Accordingly, there is a need to provide antiperspirant compositions that are in liquid form to enable spray-on or roll-on application and that exhibit a visibly distinct appearance from existing deodorant compositions and antiperspirant compositions. It is also desirable to provide antiperspirant products that contain such antiperspirant compositions. Furthermore, other desirable features and characteristics of the present invention will become apparent from the subsequent detailed description of the invention and the appended claims, taken in conjunction with the accompanying drawings and this background of the invention.

### BRIEF SUMMARY

Multi-phase liquid antiperspirant compositions and antiperspirant products including multi-phase liquid antiperspirant compositions are provided. In one embodiment, a multi-phase liquid antiperspirant composition includes a first liquid phase, a second liquid phase, and a surfactant component. The first liquid phase includes an antiperspirant active compound and water. The second liquid phase includes a cyclomethicone component. The surfactant component is present in the multi-phase liquid antiperspirant composition in an amount sufficient to form an unstable emulsion of the first liquid phase and the second liquid phase upon mixing of the multi-phase liquid antiperspirant composition. The first liquid phase and the second liquid phase separate into visibly distinct layers within the multi-phase liquid antiperspirant composition after a settling time period of less than or equal to 48 hours.

In another embodiment, a multi-phase liquid antiperspirant composition includes a first liquid phase, a second liquid phase, and a surfactant component. The first liquid phase includes an antiperspirant active compound, a buffer component, water, and a dye. The second liquid phase includes a cyclomethicone component and a fragrance. The surfactant component is present in the multi-phase liquid antiperspirant composition in an amount sufficient to form an unstable emulsion of the first liquid phase and the second liquid phase upon mixing of the multi-phase liquid antiperspirant composition. The first liquid phase and the second liquid phase separate into visibly distinct layers within the multi-phase liquid antiperspirant composition after a settling time period of less than or equal to 48 hours.

In another embodiment, an antiperspirant product includes a container and a multi-phase liquid antiperspirant composition disposed in the container. The multi-phase liquid antiperspirant composition includes a first liquid phase, a second liquid phase, and a surfactant component. The first liquid phase includes an antiperspirant active compound and water. The second liquid phase includes a cyclomethicone component. The surfactant component is present in the multi-phase liquid antiperspirant composition in an amount sufficient to form an unstable emulsion of the first liquid phase and the second liquid phase upon mixing of the multi-phase liquid antiperspirant composition. The first liquid phase and the second liquid phase separate into visibly distinct layers within the multi-phase liquid antiperspirant composition after a settling time period of less than or equal to 48 hours.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Multi-phase liquid antiperspirant compositions and antiperspirant products that include the multi-phase liquid antiperspirant compositions are provided herein. By “liquid”, it is meant that the antiperspirant compositions are flowable at ambient temperature of about 21° C. In an embodiment, the multi-phase liquid antiperspirant compositions have sufficiently low viscosity to enable spray-on and/or roll-on application. By “multi-phase”, it is meant that the antiperspirant compositions include a first liquid phase and a second liquid phase that separate into visibly distinct layers within the multi-phase liquid antiperspirant compositions after a settling time period of less than or equal to 48 hours. In particular, the first liquid phase and the second liquid phase are capable of forming an unstable emulsion upon mixing of the multi-phase liquid antiperspirant compositions, with the first liquid phase and the second liquid phase separating into visibly distinct layers within the multi-phase liquid antiperspirant compositions after the settling time period. The ability of the multi-phase liquid antiperspirant compositions to form an unstable emulsion provides multi-faceted benefits: the multi-phase liquid antiperspirant compositions may possess similar aesthetic and application properties that have made existing deodorant compositions in the form of water-based solutions commercially desirable; a substantially homogenous distribution of components within the multi-phase liquid antiperspirant compositions can be achieved on a temporary basis to ensure consistent application and performance properties of the multi-phase liquid antiperspirant compositions once applied; and the separation of the first liquid phase and the second liquid phase into visibly distinct layers within the multi-phase liquid antiperspirant compositions after the settling time period provides a basis for distinction of the multi-phase liquid antiperspirant compositions from the plethora of commercially available deodorant products and may impart a more clinical appearance as compared to stable emulsions. The viscosity of the multi-phase liquid antiperspirant compositions is sufficiently low to allow for formation of the distinct layers within the multi-phase liquid antiperspirant compositions within the settling time period to provide the unique appearance of the multi-phase liquid antiperspirant compositions.

As set forth above, the multi-phase liquid antiperspirant composition includes the first liquid phase. The first liquid phase is based upon water and/or water-miscible compounds that are liquid at ambient temperature of about 21° C. The first liquid phase includes an antiperspirant active compound and water. Optional components may also be included in the first liquid phase such as, but not limited to, a buffer component, a monohydric alcohol component, and/or dye. Because the antiperspirant active compound is present in the first liquid phase, antiperspirant efficacy of the multi-phase liquid antiperspirant composition is attributable to the first liquid phase.

Antiperspirant active compounds typically include metal salts and are thought to reduce perspiration by diffusing through the sweat ducts of eccrine glands and apocrine glands and hydrolyzing in the sweat ducts, where they combine with proteins to form an amorphous metal hydroxide agglomerate, plugging the sweat ducts so perspiration cannot diffuse to the skin surface. Examples of antiperspirant active compounds that may be used include astringent metallic salts, especially inorganic and organic salts of aluminum. In one embodiment, the antiperspirant active compound is a zirconium-free aluminum salt, which class of salts is preferred for aerosol and pump dispensing of the multi-phase liquid antiperspirant composition. Suitable zirconium-free aluminum salts include, for example, aluminum halides, aluminum chlorohydrates, aluminum hydroxyhalides, and combinations thereof. Exemplary zirconium-free aluminum salts include those having the general formula Al₂(OH)ₐCl_{b} x (H₂O), wherein a is from 2 to about 5; the sum of a and b is about 6; x is from about 1 to about 6; and wherein a, b, and x may have non-integer values. Specific examples of antiperspirant active compounds suitable for use in the various embodiments contemplated herein include aluminum dichlorohydrate, aluminum sesquichlorohydrate, aluminum chlorohydrex propylene glycol complex, aluminum dichlorohydrex propylene glycol complex, aluminum sesquichiorohydrex propylene glycol complex, aluminum chlorohydrex polyethylene glycol complex, aluminum dichlorohydrex polyethylene glycol complex, aluminum sesquichlorohydrex polyethylene glycol complex, aluminum chloride, aluminum sulfate buffered, and combinations thereof. It is to be appreciated that a combination of antiperspirant active compounds may be included in the first liquid phase. In one specific embodiment, the antiperspirant compound is aluminum sesquichlorohydrate.

In an embodiment, the antiperspirant active compound, or combination of antiperspirant active compounds, is present in the first liquid phase in an amount of about 5 to about 50 weight %, such as from about 15 to about 35 weight %, based upon the total weight of the first liquid phase. As used herein, weight percent of the antiperspirant active compound(s) is calculated as anhydrous weight percent in accordance with the U.S.P. method, as is known in the art. This calculation excludes any bound water and buffer. In one embodiment, all antiperspirant active compounds included in the multi-phase liquid antiperspirant composition are introduced therein through the first liquid phase. In the multi-phase liquid antiperspirant composition that includes the first liquid phase and the second liquid phase, the antiperspirant active compounds may be present in a total amount of from about 5 to about 25 weight %, such as from about 15 to about 20 weight %, based upon the total weight of the multi-phase liquid antiperspirant composition. In an embodiment, water is present in the first liquid phase in an amount of from about 30 to about 85 weight %, such as from about 50 to about 75 weight %, based upon the total weight of the first liquid phase.

In addition to the water, and as alluded to above, the first liquid phase may also include the monohydric alcohol component that, together with the water, functions as a carrier solvent in the first liquid phase. The monohydric alcohol component may be included to provide drying efficiency to the multi-phase liquid antiperspirant compositions. The monohydric alcohol component represents any monohydric alcohols that are present in the first liquid phase and, thus, can include one or more monohydric alcohols. Suitable monohydric alcohols that may be included in the first liquid phase are not particularly limited and may include any monohydric alcohols that are known to be included in antiperspirant compositions. In one embodiment, the monohydric alcohol component includes one or more C₁ to C₆ monols. Examples of suitable monohydric alcohols include, but are not limited to, methanol, ethanol, propanol, isopropanol, and combinations thereof, with ethanol being particularly preferred. In an embodiment, the monohydric alcohol component is present in the first liquid phase in an amount of from greater than about 0 to about 25 weight %, for example from about 55 to about 25 weight %, based upon the total weight of the first liquid phase.

The buffer component represents any buffers that are present in the first liquid phase and, thus, can include one or more buffers. Suitable buffers for antiperspirant active compounds are known in the art and can include polyhydric alcohols, amino acids, and combinations thereof. The buffers function to stabilize pH of the antiperspirant active compounds and reduce skin irritability that the antiperspirant active compounds may cause. Additionally, the buffers may contribute to activation of the antiperspirant active compounds. In one embodiment, the buffer component includes one or more amino acids. Suitable amino acids include glycine, alanine, leucine, isoleucine, β-alanine, valine, cysteine, serine, tryptophane, phenylalanine, methionine, β-amino-n-butanoic acid, γ-amino-n-butanoic acid, and salts thereof, which may be in the d-form, the i-form or the dl-form. In one specific embodiment, the buffer component includes glycine. In an embodiment, the buffer component is present in the first liquid phase in an amount of from about 0.5 to about 4.0 weight %, %, such as, for example, from about 0.7 to about 3.5 weight %, or, as another example, from about 2.0 to about 3.0 weight %, all based upon the total weight of the first liquid phase.

The first liquid phase may be prepared through any known method of forming a water-based antiperspirant composition that is known in the art. In one embodiment, to achieve excellent clarity of the first liquid phase, the first liquid phase may be prepared by providing an antiperspirant active component that includes one or more antiperspirant active compounds, water, and a first buffer. In this embodiment, the antiperspirant active component may be mixed with additional water, if a desired full charge of water is not already present in the antiperspirant active component, and a second buffer to form a first mixture. The first mixture is heated either during or after formation of the first mixture, for example to a temperature of at least about 50° C. The first buffer and the second buffer may be the same or different and may include any of those mentioned above. In one embodiment, the first buffer is present in the antiperspirant active component prior to mixing to form the first mixture in an amount of from about 0.5 to about 2.1 weight %, such as from about 1.0 to about 2.1 weight %, based upon the total weight of the antiperspirant active component. Based upon the amount ranges of the first buffer in the antiperspirant active component, as set forth above, and further based upon the relative amounts of water and antiperspirant active compounds present in the antiperspirant active component, the amount of first buffer present in the first liquid phase can be from about 0.05 to about 1.5 weight %, such as from about 0.1 to about 1.0 weight %, based upon the total weight of the first liquid phase. In an embodiment, the second buffer is mixed with the antiperspirant active component and water in an amount of from about 0.4 to about 3.8 weight %, such as from about 0.4 to about 1.5 weight %, with the first and the second buffers present in the first mixture in a combined amount of from about 0.5 to about 4.0 weight based upon the total weight of the first liquid phase. In this embodiment, any monohydric alcohol to be included in the first liquid phase is then mixed with the first mixture to form the first liquid phase. In effect, this particular method involves back-loading additional buffer to achieve a higher buffer content in the first mixture than may be initially present in the antiperspirant active component which, in combination with heating, can result in excellent clarity of the first liquid phase. However, it is to be appreciated that under some circumstances, excellent clarity may not be desired. For example, an opaque appearance of the first liquid phase may be desired to provide a distinct appearance from the second liquid phase.

As set forth above, the multi-phase liquid antiperspirant composition includes the second liquid phase. The second liquid phase includes a cyclomethicone component, which is immiscible with water at ambient temperature of about 21° C. Optional components may also be included in the second liquid phase such as, but not limited to, a fragrance, a fragrance emulsifier, and/or dye. The second liquid phase provides numerous benefits to the multi-phase liquid antiperspirant composition. In addition to enabling the visibly distinct layers to be formed in the multi-phase liquid antiperspirant compositions and, thus, providing appearance benefits to the multi-phase liquid antiperspirant compositions, the presence of the cyclomethicone component in the second liquid phase also provides drying efficiency to the multi-phase liquid antiperspirant compositions and thereby obviates the primary function of monohydric alcohols that may be included in the first liquid phase. Further, the cyclomethicone component provides an excellent carrier solvent for the fragrance (when included).

The cyclomethicone component represents any cyclomethicones that are present in the second liquid phase and, thus, can include one or more cyclomethicones. Exemplary cyclomethicones include cyclotetrasiloxane, cyclopentasiloxane, cyclohexasiloxane, and combinations thereof. In one specific embodiment, the cyclomethicone component includes a combination of cyclotetrasiloxane and cyclopentasiloxane. In an embodiment, the cyclomethicone component is present within the second liquid phase in an amount of from about 70 to about 100 weight %, for example, from about 80 to about 99 weight %, based upon the total weight of the antiperspirant composition.

Fragrances that are suitable for antiperspirant compositions are well known in the art and are typically oily in nature. Suitable fragrance emulsifiers include nonionic emulsifiers such as, but not limited to, polysorbates, polyalkylene glycols, fatty alcohols, and mixtures thereof. Preferred nonionic emulsifiers for use herein include alkylphenol ethers; linear aliphatic or fatty C4-C16 alcohols; mono- and di-esters of aliphatic C4-C16 carboxylic acids; branched aliphatic alcohols with a main aliphatic carbon chains of C4-C16; hydrogenated castor oils (such as those available under the tradename Cremophor® from BASF Corporation of Florham Park, New Jersey) and glycols; ethoxylated hydrogenated castor oil monopyroglutamic monoisostearic diesters; ethoxylated glycerol monopyroglutamic moniostearic diesters; and combinations thereof.

In one embodiment, the fragrance is present in the second liquid phase in an amount of from about 0.1 to about 2.0 weight %, such as from about 0.1 to about 0.7 weight %, based upon the total weight of the second liquid phase. In one embodiment, the fragrance emulsifier is present in the second liquid phase in an amount of from about 0.1 to about 2.0 weight %, such as from about 0.5 to about 1.0 weight %, based upon the total weight of the second liquid phase.

The second liquid phase may be prepared through any known method of forming a cyclomethicone-based antiperspirant composition that is known in the art. In an embodiment, a fragrance premix is first prepared including the fragrance and the fragrance emulsifier, followed by mixing the cyclomethicone component and the fragrance premix. However, it is to be appreciated that all components of the second liquid phase may be simultaneously mixed.

As alluded to above, the first liquid phase and/or the second liquid phase may further include a dye. In an embodiment, at least one of the first liquid phase and the second liquid phase includes the dye. The dye may enhance or serve as a basis for visual distinctiveness of the layers within the multi-phase liquid antiperspirant compositions after the settling time period. However, it is to be appreciated that the first liquid phase and the second liquid phase may be visibly distinct without the use of dye. For example, one of the first liquid phase or the second liquid phase could be more opaque than the other of the liquid phases, thereby providing visible distinction through a modulation in light transparent between the first liquid phase and the second liquid phase. When used, appropriate dyes could be selected based upon the nature of the particular liquid phase in which the dyes are to be included, i.e., the water-based first liquid phase or the cyclomethicone-based second liquid phase. The dye may be present in the first liquid phase and/or the second liquid phase in an amount of from about 0.1 to about 2.0 weight %, such as from about 0.1 to about 0.5 weight %, based upon the total weight of the particular liquid phase in which the dye is included.

As set forth above, the multi-phase liquid antiperspirant compositions include the first liquid phase and the second liquid phase that are capable of forming an unstable emulsion upon mixing of the multi-phase liquid antiperspirant compositions, meaning that the first liquid phase and the second liquid phase are capable of forming a temporary emulsion that visibly appears as a homogeneous solution but that eventually separates into visibly distinct layers. Emulsion stability may also be signified by higher viscosities, whereas unstable emulsions generally have comparably lower viscosities. In an embodiment, the unstable emulsion of the first liquid phase and the second liquid phase upon mixing of the multi-phase liquid antiperspirant composition has a viscosity of no greater than about 1000 centipoise, such as no greater than about 500 centipoise or no greater than about 50 centipoise, at 25° C. For example, the unstable emulsion of the first liquid phase and the second liquid phase upon mixing of the multi-phase liquid antiperspirant composition can have a viscosity of from about 1 to about 1000 centipoise, such as from about 1 to about 500 centipoise or from about 1 to about 50 centipoise, at 25° C.

To enable the first liquid phase and the second liquid phase to form the unstable emulsion, the multi-phase liquid antiperspirant compositions include a surfactant component. The surfactant component represents any surfactants that are included in the multi-phase liquid antiperspirant composition. In this regard, the surfactant component includes the fragrance emulsifier when the fragrance emulsifier is present in the second liquid phase. When the fragrance emulsifier is present in the second liquid phase, an additional surfactant or surfactants may be included in the multi-phase liquid antiperspirant composition for purposes of achieving the unstable emulsion of the first liquid phase and the second liquid phase. Alternatively, the fragrance emulsifier may enable the unstable emulsion of the first liquid phase and the second liquid phase to be achieved without the need for additional surfactants.

To enable formation of the unstable emulsion of the first liquid phase and the second liquid phase, various factors may be considered such as chemistry and HLB of the surfactants in the surfactant component and amount of the surfactant component in the multi-phase liquid antiperspirant composition. Those of skill in the art, in combination with the descriptions herein, can readily identify an appropriate combination of such factors to achieve an unstable emulsion of the first liquid phase and the second liquid phase in the multi-phase liquid antiperspirant compositions described herein. Notably, chemistries, HLBs, and amounts of the surfactant component are chosen to avoid formation of a stable emulsion (i.e., an emulsion that will not separate after a settling time period of at least 48 hours) of the first liquid phase and the second liquid phase in the multi-phase liquid antiperspirant compositions described herein.

Distinct from the fragrance emulsifiers mentioned above, suitable surfactants that may be included in the surfactant component may include hydrocarbon-containing surfactants, silicone-containing surfactants, and combinations thereof By “hydrocarbon-containing” or “silicone-containing”, it is meant that the surfactants are derived from hydrocarbons or silicones and that a portion of the chemical structure in the resulting surfactant can be identified as a hydrocarbon or silicone. In one embodiment, the surfactant component includes a silicone-containing surfactant. Silicone-containing surfactants have been found to produce relatively clear visibly distinct layers that include the first liquid phase and the second liquid phase in the multi-phase liquid antiperspirant compositions and, thus, may provide particular aesthetic benefits to the multi-phase liquid antiperspirant compositions as compared to other types of surfactants. Specific examples of suitable silicone-containing surfactants include, but are not limited to, polysiloxane polyalkylether copolymers such as cetyl polysiloxane PEG/PPG-10/1 copolymers; bis-PEG/PPG-20/5PEG/PPG-20/5 dimethicone, methoxy PEG/PPG-25/4 dimethicone, caprylic/caprictriglyceride copolymers; and PEG/PPG-14/4 dimethicone. Examples of suitable hydrocarbon-containing surfactants include, but are not limited to, alkyleneoxy adducts of fatty acids such as PEG-20 stearyl ether, PEG-7 glyceryl cocoate; alkyleneoxy and fatty acid adducts of sorbitan such as polysorbate 20; alkyl glycosides such as decyl glucoside; amide-containing compounds such as palmitamidopropyltrimonium chloride; and combinations thereof. The hydrocarbon-containing surfactants each have a hydrocarbon chain in the chemical structure thereof to provide the hydrophobic functionality, whereas the silicone-containing surfactants have a siloxane chain in the chemical structure thereof to provide the hydrophobic functionality. In an embodiment, the surfactant component (and, thus, the multi-phase liquid antiperspirant composition) is free of ionic surfactants. Stated differently, in this embodiment, the surfactant component only includes non-ionic surfactants.

Within the above-described categories of suitable surfactants that may be included in the surfactant component, in an embodiment, the surfactants also free from surfactants having an HLB value of from about 8 to about 10. In particular, surfactants having an HLB value of from about 8 to about 10 generally promote formation of a stable emulsion between water-based phases and cyclomethicone-based phases and, as such, may impact the ability to achieve an unstable emulsion as desired for the multi-phase liquid antiperspirant compositions described herein. In this embodiment, the surfactants that are included in the surfactant component may have an HLB of less than 8, such as from 3 up to 8 or from 5 to 7. Alternatively, the surfactants that are included in the surfactant component may have an HLB of greater than 10, such as from greater than 10 to about 17 or from about 11 to about 16. Of course, a combination of surfactants may be present in the surfactant component, with one or more surfactants having an HLB of less than 8 and one or more surfactants having an HLB of greater than 10.

The surfactant component is present in the multi-phase liquid antiperspirant composition in an amount sufficient to form an unstable emulsion of the first liquid phase and the second liquid phase upon mixing of the multi-phase liquid antiperspirant composition. It is to be appreciated that different surfactants have a different effect on the ability to achieve a stable emulsion based upon the amount of the particular surfactants within the multi-phase liquid antiperspirant composition, and the amounts of such surfactants are below the thresholds that would enable a stable emulsion to be achieved. In an embodiment, the surfactant component is present in an amount of from about 0.1 to about 5.0 weight %, such as from about 0.1 to about 3.0, based upon the total weight of the multi-phase liquid antiperspirant composition.

The surfactant component may be introduced through the first liquid phase, the second liquid phase, or may be introduced independent from the first liquid phase and the second liquid phase. In one specific embodiment, the surfactant component is included in the second liquid phase prior to mixing the second liquid phase and the first liquid phase.

The first liquid phase and the second liquid phase may be present in the multi-phase liquid antiperspirant compositions in such amounts that the distinct layers of the first liquid phase and the second liquid phase each have a thickness of at least 10% of a total combined thickness of the distinct layers within the multi-phase liquid antiperspirant composition after the settling time period. In this regard, the amount of the first liquid phase to the second liquid phase may be from about 10 to 1 to about 1 to 10. In an embodiment, the surfactant component may form another distinct layer within the multi-phase liquid antiperspirant compositions to provide a tri-layer appearance to the multi-phase liquid antiperspirant compositions. However, the surfactant component is typically contained within the first liquid phase or the second liquid phase and does not separate therefrom over the settling time period. Further, it is to be appreciated that components described above that are within the first liquid phase, the second liquid phase, and/or the surfactant component may be present within one or more of the other phases in small quantities, although not in sufficient quantities to render a homogeneous appearance to the multi-phase liquid antiperspirant compositions. Stated differently, the multi-phase liquid antiperspirant compositions still have visibly distinct layers after the settling time period. In an embodiment, a distinct barrier exists between the visibly distinct layers. In another embodiment, a barrier between the visibility distinct layers is non-defined, with a transition zone located between the visibly distinct layers in which the distinct layer containing the first liquid phase transitions to the distinct layer containing the second liquid phase.

As set forth above, due to the formation of the unstable emulsion upon mixing of the multi-phase liquid antiperspirant compositions, the first liquid phase and the second liquid phase separate into visibly distinct layers within the multi-phase liquid antiperspirant compositions after the settling time period. In particular, the first liquid phase and the second liquid phase separate into visibly distinct layers within the multi-phase liquid antiperspirant composition after a settling time period of less than or equal to 48 hours, such as from one minute to about 24 hours, from 1 minute to 12 hours, or from 1 minute to 2 hours. The mixing of the multi-phase liquid antiperspirant compositions to achieve the unstable emulsion may involve shaking a container including the multi-phase liquid antiperspirant composition therein, mixing the multi-phase liquid antiperspirant composition in a vessel, etc. until visibly homogeneous. Separation into the visibly distinct layers may be measured at ambient temperature of about 21° C. after leaving the multi-phase liquid antiperspirant compositions undisturbed.

In an embodiment, an antiperspirant product is provided that includes a container and the multi-phase liquid antiperspirant composition as described above. More specifically, the multi-phase liquid antiperspirant composition is disposed within the container, which can be any container that is acceptable for sale to consumers and that is capable of retaining a liquid such as a spray bottle or roll-on container.

### EXAMPLES

Multi-phase liquid antiperspirant compositions were prepared to evaluating the effect of various surfactants on the ability to achieve an unstable emulsion in the multi-phase liquid antiperspirant compositions. To prepare the multi-phase liquid antiperspirant compositions, a first liquid phase and a second liquid phase are separately prepared. Prior to mixing the first liquid phase and the second liquid phase, a surfactant component is mixed with the second liquid phase. Finally, the first liquid phase and the second liquid phase that includes the surfactant component are mixed to provide the multi-phase liquid antiperspirant compositions.

### Preparation 1

The first liquid phase is prepared by providing an antiperspirant active component that includes the antiperspirant active compound, water, and a first buffer. The antiperspirant active component is mixed with additional water and a second buffer to form a first mixture. The first mixture is heated to a temperature of about 65° C. and held at temperature for about 10 minutes, followed by cooling to a temperature of below about 40° C. Dye is then added to the first mixture to form the first liquid phase. The second liquid phase includes about 100 weight % of cyclopentasiloxane, based upon the total weight of the second liquid phase. Amounts of each ingredient in the first liquid phase and the second liquid phase are set forth in Table I below, with the amounts in weight %, based upon the total weight of the respective phase in which the ingredient is included.

TABLE I

| | | | Ref. |
|---|---|---|---|
| | | | Ex. 1 |
| First | | | |
| Liquid Phase | | | |
| | Antiperspirant Active | | |
| | Component | | |
| | | Antipersp. Active | 29.93 |
| | | Compound | |
| | | Water | 64.57 |
| | | First Buffer | 2.00 |
| | Additional Water | | 1.00 |
| | Total Water | | 65.57 |
| | Second Buffer | | 1.50 |
| | Total Buffer | | 3.50 |
| | Dye | | 1.00 |
| | Total Weight, First | | 100.00 |
| | Liquid Phase | | |
| Second | | | |
| Liquid Phase | | | |
| | Cyclopentasiloxane | | 100.00 |

Antiperspirant Active Compound is aluminum sesquichlorohydrate (AASC).

First Buffer and Second Buffer are glycine.

Dye is Blue 1.

### EXAMPLE 1

As set forth above, prior to mixing the first liquid phase and the second liquid phase of Reference Example 1, a surfactant component is mixed with the second liquid phase in an amount of 2 weight % surfactant component to 98 weight % of the second liquid phase, based upon the total combined weight of the surfactant component and the second liquid phase. The mixture of the surfactant component and the second liquid phase was subjected to turbulent mixing for about 1 minute. The first liquid phase and the mixtures including the surfactant component and the second liquid phase were mixed at a 1:1 weight ratio, effectively providing multi-phase liquid antiperspirant compositions having about 50 weight % of the first liquid phase, about 49 weight % of the second liquid phase, and about 1 weight % of the surfactant component. The multi-phase liquid antiperspirant compositions were subjected to turbulent mixing for about 10 seconds in 20 ml disposable scintillation vials and then set on a flat surface to evaluate phase separation within the multi-phase liquid antiperspirant compositions. Phase separation was defined as when one phase separates ⅛″ from another phase as determined through visual observation. The various types of surfactant components and amounts thereof are set forth in Table II below, with the amounts based upon the total weight of the multi-phase liquid antiperspirant composition unless indicated otherwise. Settling time periods at which visibly distinct layers are achieved in the multi-phase liquid antiperspirant compositions are set forth in Table II for various combinations of the first liquid phase, the second liquid phase, and surfactant components.

TABLE II

| | Ex. 1 | Ex. 2 | Ref. Ex. 3 | Ref. Ex. 4 | Ref. Ex. 5 |
|---|---|---|---|---|---|
| First | 50.00 | 50.00 | 50.00 | 50.00 | 50.00 |
| Liquid | | | | | |
| Phase | | | | | |
| Second | 49.00 | 49.00 | 49.00 | 49.00 | 49.00 |
| Liquid | | | | | |
| Phase | | | | | |
| Surfactant | 1.00 | — | — | — | — |
| A | | | | | |
| Surfactant | — | 1.00 | — | — | — |
| B | | | | | |
| Surfactant | — | — | 1.00 | — | — |
| C | | | | | |
| Surfactant | — | — | — | 1.00 | — |
| D | | | | | |
| Surfactant | — | — | — | — | 1.00 |
| E | | | | | |
| Surfactant | — | — | — | — | — |
| F | | | | | |
| Surfactant | — | — | — | — | — |
| G | | | | | |
| Surfactant | — | — | — | — | — |
| H | | | | | |
| Surfactant | — | — | — | — | — |
| J | | | | | |
| Total | 100.00 | 100.00 | 100.00 | 100.00 | 100.00 |
| Settling | 1.5 | 16 | 6 | 1 | 20 |
| Time | | | | | |
| Period, | | | | | |
| minutes | | | | | |
| Obser- | First | Second | Three | Three | First |
| vations | Liquid | Liquid | Distinct | Distinct | Liquid |
| | Phase | Phase | Layers; | Layers; | Phase |
| | Separates | Separates | Surfactant | Surfactant | Separates |
| | First | First | Interlayer | Interlayer | First |
| | Ref. Ex. 6 | Ref. Ex. 7 | Ref. Ex. 8 | Ref. Ex. 9 | Comp. Ex. |
| First | 50.00 | 50.00 | 50.00 | 50.00 | 50.00 |
| Liquid | | | | | |
| Phase | | | | | |
| Second | 49.00 | 49.00 | 49.00 | 49.00 | 50.00 |
| Liquid | | | | | |
| Phase | | | | | |
| Surfactant | — | — | — | — | — |
| A | | | | | |
| Surfactant | — | — | — | — | — |
| B | | | | | |
| Surfactant | — | — | — | — | — |
| C | | | | | |
| Surfactant | — | — | — | — | — |
| D | | | | | |
| Surfactant | — | — | — | — | — |
| E | | | | | |
| Surfactant | 1.00 | — | — | — | — |
| F | | | | | |
| Surfactant | — | 1.00 | — | — | — |
| G | | | | | |
| Surfactant | — | — | 1.00 | — | — |
| H | | | | | |
| Surfactant | — | — | — | 1.00 | — |
| J | | | | | |
| Total | 100.00 | 100.00 | 100.00 | 100.00 | 100.00 |
| Settling | 7.75 | 28 | 58 | 11.75 | 0 |
| Time | | | | | |
| Period, | | | | | |
| Minutes | | | | | |
| Obser- | First | First | First | First | Immediate |
| vations | Liquid | Liquid | Liquid | Liquid | Phase |
| | Phase | Phase | Phase | Phase | Separation |
| | Separates | Separates | Separates | Separates | |
| | First | First | First | First | |

Surfactant A is a cetyl polysiloxane PEG/PPG-10/1 copolymer surfactant commercially available from Goldschmidt Chemical Corporation of Hopewell, Virginia.

Surfactant B is a bis-PEG/PPG-20/5PEG/PPG-20/5 dimethicone, methoxy PEG/PPG-25/4 dimethicone, caprylic/caprictriglyceride copolymer surfactant commercially available from Goldschmidt Chemical Corporation.

Surfactant C is a PEG-40 hydrogenated castor oil surfactant commercially available from BASF Corporation of Florham Park, N.J.

Surfactant D is a PEG/PPG-14/4 dimethicone copolymer surfactant commercially available from Goldschmidt Chemical Corporation.

Surfactant E is a decyl glucoside surfactant commercially available from Cognis Corporation of Ambler, Pa.

Surfactant F is a palmitamidopropyltrimonium chloride surfactant commercially available from Goldschmidt Chemical Corporation.

Surfactant G is a PEG-20 stearyl ether copolymer surfactant commercially available from Sigma-Aldrich Corporation of St. Louis, Mo.

Surfactant H is a PEG-7 glyceryl cocoate surfactant commercially available from Cognis Corporation.

Surfactant J is a polysorbate 20 surfactant commercially available from Sigma-Aldrich Corporation.

With each of the Examples, the multi-phase liquid antiperspirant compositions separated into visibly distinct layers including the first liquid phase and the second liquid phase, respectively, with Examples 3 and 4 forming a third phase including the surfactant component as an interlayer between layers that include the first liquid phase and the second liquid phase.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A multi-phase liquid antiperspirant composition comprising:
a first liquid phase comprising:
an antiperspirant active compound; and
water,
a second liquid phase comprising a cyclomethicone component; and
a surfactant component present in said multi-phase liquid antiperspirant composition in an amount sufficient to form an unstable emulsion of said first liquid phase and said second liquid phase upon mixing of said multi-phase liquid antiperspirant composition;
wherein said first liquid phase and said second liquid phase separate into visibly distinct layers within said multi-phase liquid antiperspirant composition after a settling time period of less than or equal to 48 hours.

2. The multi-phase liquid antiperspirant composition of claim 1, wherein said surfactant component is free of ionic surfactants.

3. The multi-phase liquid antiperspirant composition of claim 1, wherein said surfactant component is free from surfactants having an HLB value of from about 8 to about 10.

4. The multi-phase liquid antiperspirant composition of claim 1, wherein said surfactant component comprises a silicone-containing surfactant.

5. The multi-phase liquid antiperspirant composition of claim 1, wherein said surfactant component is present in an amount of from about 0.1 to about 5.0 weight %, based upon the total weight of said multi-phase liquid antiperspirant composition.

6. The multi-phase liquid antiperspirant composition of claim 1, wherein said distinct layers of said first liquid phase and said second liquid phase each have a thickness of at least 10% of a total combined thickness of said visibly distinct layers within said multi-phase liquid antiperspirant composition after the settling time period.

7. The multi-phase liquid antiperspirant composition of claim 1, wherein the settling time period is from about 1 minute to about 24 hours.

8. The multi-phase liquid antiperspirant composition of claim 1, wherein said antiperspirant active compound is a zirconium-free aluminum salt.

9. The multi-phase liquid antiperspirant composition of claim 1, wherein the unstable emulsion of said first liquid phase and said second liquid phase upon mixing of said multi-phase liquid antiperspirant composition has a viscosity of no greater than 1000 centipoise at 25° C.

10. The multi-phase liquid antiperspirant composition of claim 1, wherein said water is present in said first liquid phase in a total amount of from about 30 to about 85 weight %, based upon the total weight of said first liquid phase.

11. The multi-phase liquid antiperspirant composition of claim 1, wherein:
said antiperspirant active compound is present in said first liquid phase in an amount of about 5 to about 50 weight %,
said water is present in said first liquid phase in an amount of from about 50 to about 85 weight %, and
all based upon the total weight of said first liquid phase.

12. The multi-phase liquid antiperspirant composition of claim 1, wherein said first liquid phase further comprises a buffer component.

13. The multi-phase liquid antiperspirant composition of claim 1, wherein said second liquid phase further comprises a fragrance.

14. The multi-phase liquid antiperspirant composition of claim 1, wherein said first liquid phase further comprises a monohydric alcohol component.

15. The multi-phase liquid antiperspirant composition of claim 1, wherein at least one of said first liquid phase and said second liquid phase comprises a dye.

16. A multi-phase liquid antiperspirant composition comprising:
a first liquid phase comprising:
an antiperspirant active compound;
a buffer component;
water; and
a dye;
a second liquid phase comprising:
a cyclomethicone component; and
a fragrance; and
a surfactant component present in said multi-phase liquid antiperspirant composition in an amount sufficient to form an unstable emulsion of said first liquid phase and said second liquid phase upon mixing of said multi-phase liquid antiperspirant composition;
wherein said first liquid phase and said second liquid phase separate into visibly distinct layers within said multi-phase liquid antiperspirant composition after a settling time period of less than or equal to 48 hours.

17. The multi-phase liquid antiperspirant composition of claim 16, wherein:
said antiperspirant active compound is present in said first liquid phase in an amount of about 5 to about 50 weight %;
said buffer component is present in said first liquid phase in an amount of from about 0.5 to about 4.0 weight %;
said water is present in said first liquid phase in an amount of from about 50 to about 85 weight %, and
said dye is present in said first liquid phase in an amount of from about 0.1 to about 2.0 weight %,
all based upon the total weight of said first liquid phase.

18. The multi-phase liquid antiperspirant composition of claim 16, wherein said surfactant component is free of ionic surfactants.

19. The multi-phase liquid antiperspirant composition of claim 16, wherein said surfactant component is free from surfactants having an HLB value of from about 8 to about 10.

20. An antiperspirant product comprising:
a container; and
a multi-phase liquid antiperspirant composition disposed in said container, said multi-phase liquid antiperspirant composition comprising:
a first liquid phase comprising:
an antiperspirant active compound; and
water,
a second liquid phase comprising a cyclomethicone component; and
a surfactant component present in said multi-phase liquid antiperspirant composition in an amount sufficient to form an unstable emulsion of said first liquid phase and said second liquid phase upon mixing of said multi-phase liquid antiperspirant composition;
wherein said first liquid phase and said second liquid phase separate into visibly distinct layers within said multi-phase liquid antiperspirant composition after a settling time period of less than or equal to 48 hours.
